(19) United States
(12) Patent Application Publication
(10) Pub. No.: US 2016/0030670 A1
(43) Pub. Date: Feb. 4, 2016

(54) Blood Glucose and Insulin Control Systems and Methods

(71) Applicant: The General Hospital Corporation, Boston, MA (US)

(72) Inventor: Bruce Fischl, Boston, MA (US)

(21) Appl. No.: 14/815,502

(22) Filed: Jul. 31, 2015

Related U.S. Application Data
(60) Provisional application No. 62/032,112, filed on Aug. 1, 2014.

(51) Int. Cl. A61M 5/172; A61M 5/142

(52) U.S. Cl. CPC A61M 5/1723; A61M 5/14244; A61M 2005/14208; A61M 2005/14268; A61M 2005/1726; A61M 2230/201

(57) ABSTRACT

Embodiments of the invention provide a system and method for administering a pharmaceutical to an individual through a dispenser control system. Input data is acquired from an input device coupled to a controller of the dispenser control system. The controller is in communication with the input device and configured to execute a stored program to calibrate pump settings of the dispenser control system based on the acquired input data. The stored program also computes a delivery schedule based on the pump settings for the individual and activates the dispenser control system to deliver at least one dose of the pharmaceutical according to the delivery schedule. The delivery schedule is characterized by a waveform other than a square-wave.

### CROSS REFERENCE TO RELATED APPLICATIONS

This application claims priority from U.S. Patent Application No. 62/032,112 filed Aug. 1, 2014.

### STATEMENT REGARDING FEDERALLY SPONSORED RESEARCH

Not applicable.

### FIELD OF THE INVENTION

The present invention relates to diabetes management. More specifically, the present invention relates to systems and methods for automatically calibrating insulin pumps, developing time-varying insulin dosing schedules, and automatic minimization of postprandial blood glucose levels.

### BACKGROUND OF THE INVENTION

The pancreas of a healthy person produces and releases insulin into the blood stream in response to elevated blood plasma glucose levels. Beta cells (β-cells), which reside in the pancreas, produce and secrete the insulin into the blood stream, as it is needed. If β-cells become incapacitated or die, a condition known as Type I diabetes mellitus (or in some cases if β-cells produce insufficient quantities of insulin, Type II diabetes), then insulin must be provided to the body from another source.

Traditionally, insulin has been injected with a syringe. More recently, use of insulin pump therapy has been increasing, especially for delivering insulin for diabetics. The successful management of Type 1 diabetes depends on the ability to accurately calibrate basal and bolus insulin doses, and minimize the time spent with high postprandial blood glucose (BG) levels. The Juvenile Diabetes Research Foundation (JDRF) estimates that there are over 3 million Americans with Type 1 diabetes, with approximately 40%, or 1.4 million, of them using insulin pumps. Typically, the insulin pump is an external insulin pump worn on a belt, in a pocket, or the like, and delivers insulin into the body via an infusion tube with a percutaneous needle or a cannula placed in the subcutaneous tissue.

Currently, people with Type I or Type II diabetes that use insulin pumps must calibrate the pumps themselves. This is a complex and error-prone process as the various settings on the pump, such as basal rates of insulin delivery, carbohydrate-to-insulin ratios and sensitivity factors (i.e., the amount that blood glucose level is reduced by the bolus of a unit of insulin) vary over the course of the day by considerable amounts. In addition, these factors interact so it is difficult for a user of an insulin pump to determine which of these parameters are set incorrectly, giving rise to high or low blood glucose levels.

As such, insulin pump calibration can be difficult, particularly in young children in whom frequent and extended fasting periods are problematic. Even with fasting, setting of basal insulin levels is complicated by the long time delays between insulin administration and its effects on blood glucose levels. While correctly calibrated insulin pumps can help with successful disease management, people with Type I diabetes may experience prolonged, potentially damaging blood glucose levels due to postprandial highs, as postprandial periods constitute most of our waking hours. Thus, modulating the shape of pre-meal insulin dosing to reduce postprandial blood glucose levels and minimize the likelihood of dangerously low blood glucose levels is difficult to achieve using conventional insulin pumps.

The current standard-of-care in pump calibration uses summary statistics compiled over populations to set pump constants based largely on body weight. However, intrinsic, unaccounted for variability in individuals can result in errors in the settings of the pump that are typically in the range of 20-30%. This type of variability can have a large negative impact on average blood glucose levels. For example, a 25% error in a basal insulin can cause blood glucose levels to rise to over 200 mg/dL or drop below 70 mg/dL in just a few hours. As such, it is important for a patient's quality of life that insulin pump parameters be set more accurately than is possible based on population models.

However, with the advent of continuous glucose monitoring (CGM) in interstitial fluid, there has been a large amount of research focused on developing closed-loop systems for measuring blood glucose and delivering appropriate insulin dosages. These systems have been shown to be effective in both in-patient and out-patient studies. Further increases in control can be obtained if a soluble stable form of glucagon can be synthesized, allowing the control systems to both “push” and “pull” on blood glucose levels. For example, one recent study showed that average blood glucose levels could be reduced from 178 to 150 or so using a closed loop bi-hormonal control system.

While closed-loop control holds great long-term potential for managing Type I diabetes, open loop control has received far less attention. This is due to the great appeal of a closed-loop system, which would free patients with Type I diabetes from the burden of constant monitoring and frequent insulin dosing. However, open loop systems can be implemented much more rapidly, without requiring advances in sensor technology or stable soluble glucagon. In fact, open-loop systems are the current standard-of-care in Type I diabetes, but there has been almost no effort devoted to using optimal control theory to shape the blood glucose response to insulin dosing.

Currently, insulin doses are typically only given in two component waveforms, namely, a delta function “bolus”, or a square-wave over time (both for basal insulin, as well as for boluses to cover slow-acting carbohydrates, such as meals with high fat content). However, neither of these dosage forms is optimal for controlling postprandial blood sugar, which is the blood glucose level taken approximately two hours after a meal and used to see if someone with diabetes is taking the right amount of insulin. These waveforms for delivering insulin doses also ignore an important advantage that insulin pumps possess over injections. That is, insulin pumps could be configured to modulate the amplitude and scheduling of insulin delivery. Thus, with the advent of sophisticated and accurate mathematical models of the insulin-glucose system in Type I diabetes, it would be desirable derive insulin amplitudes and time courses that are explicitly designed to minimize hypoglycemic and hyperglycemic excursions from the target blood glucose range.

Although the insulin pump has improved the way insulin has been delivered, the insulin pump is limited in its ability to replicate all of the functions of the pancreas. Specifically, the insulin pump is still limited to delivering insulin based on user inputted commands and parameters and therefore there is a need to improve the pump to better simulate a pancreas based on current glucose values, as well as reducing postprandial blood glucose levels.

### SUMMARY OF THE INVENTION

The present invention relates to insulin pumps having integrated algorithms that reduce average blood glucose levels in Type I diabetes patients by approximately 50-70 mg/dL or more, corresponding to a two percentage point drop in A1C, a test commonly performed to diagnose Type 1 and Type 2 diabetes. In some embodiments, the integrated algorithms are used to automatically calibrate insulin pumps from measurements of individual responses to glucose and insulin, or for delivering complex insulin delivery schedule waveforms designed to keep postprandial blood glucose levels within a normal range.

Some embodiments of the invention provide a method for administering a pharmaceutical to an individual through a dispenser control system. Input data is acquired from an input device coupled to a controller of the dispenser control system. The controller is in communication with the input device and configured to execute a stored program to calibrate pump settings of the dispenser control system based on the acquired input data. The stored program also computes a delivery schedule based on the pump settings for the individual and activates the dispenser control system to deliver at least one dose of the pharmaceutical according to the delivery schedule. The delivery schedule is characterized by a waveform other than a square-wave.

Other embodiments of the invention provide a dispenser control system for administering a pharmaceutical to an individual comprising. The dispenser control system includes an input device coupled to a controller for receiving input data from the individual. A dispenser is in communication with the controller. The dispenser includes a pump for delivering the pharmaceutical from a reservoir to the individual through a flexible tubing coupled to the individual. The controller is configured to execute a program stored in the controller to calibrate pump settings of the dispenser based on the acquired input data. The stored program also computes a delivery schedule based on the pump settings for the individual and activate the dispenser control system to deliver at least one dose of the pharmaceutical according to the delivery schedule. The delivery schedule is characterized by a waveform other than a square-wave.

Other embodiments of the invention provide a method for administering a pharmaceutical to an individual through a dispenser control system. The method includes acquiring input data from at least one of a sensor coupled to the individual and an input device coupled to a controller. The controller is in communication with the sensor and configured to execute a stored program to calibrate pump settings of the dispenser control system based on the acquired input data. The stored program is also configured to compute at least one dose of the pharmaceutical to be delivered to the individual through the dispenser control system and activate the dispenser control system to deliver the at least one dose of the pharmaceutical to the individual when at least one of the pump settings and the input data is outside a predetermined threshold.

These and other features, aspects, and advantages of the present invention will become better understood upon consideration of the following detailed description, drawings, and appended claims.

### BRIEF DESCRIPTION OF THE DRAWINGS

FIG. 1 is a schematic of an example insulin pump device according to one embodiment of the present invention.

FIG. 2 is a flow chart setting forth the steps of a method for administering a pharmaceutical to an individual through a computed delivery schedule in accordance with the present invention.

FIG. 3 is a graph illustrating a blood glucose response to the infusion of insulin followed by ingestion of carbohydrates according to one computed delivery schedule.

FIG. 4 is a graph illustrating a blood glucose response to the infusion of glucagon followed by ingestion of carbohydrates according to one computed delivery schedule.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

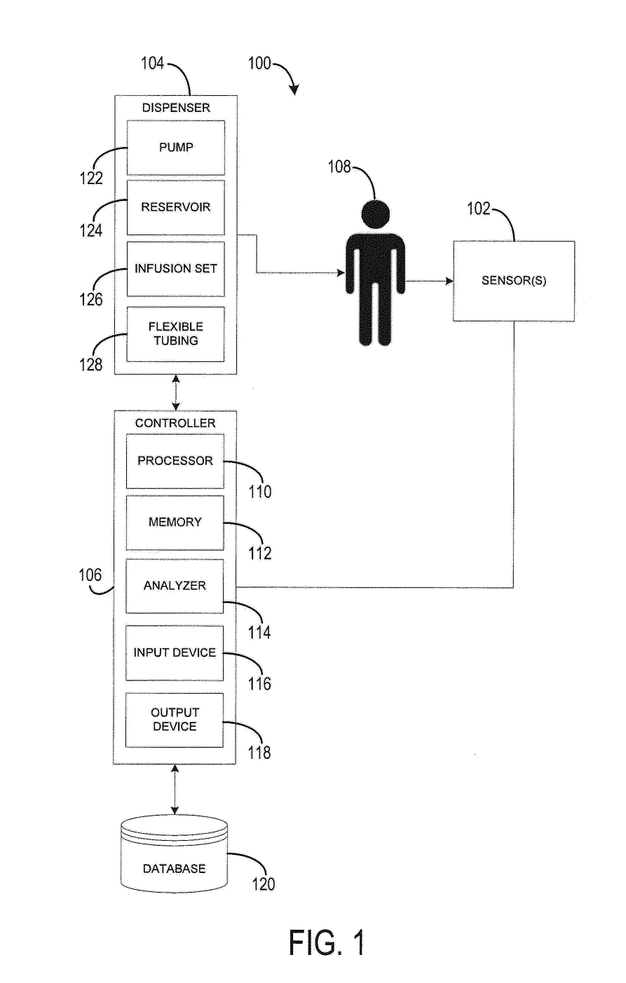

FIG. 1 illustrates an example dispenser control system 100 to control the dispensing of a pharmaceutical, such as insulin. The dispenser control system 100 includes one or more sensors 102, a dispenser 104 and a controller 106. The components of the dispenser control system 100 are communicatively linked together through communication links which may comprise, for example, radio-frequency communication links, a bus system, one or more wires, an infrared communication link, or combinations of various communication links.

The one or more sensors 102 may be a blood glucose level sensor configured to sense one or more indications of a blood glucose level of a patient 108, which may be done using direct or indirect measurement of blood glucose levels. The sensor 102 is coupled to the controller 106, which may be a blood glucose controller.

The blood glucose controller 106 includes a control system, which as illustrated comprises a processor 110, a memory 112, a blood glucose analyzer 114, an input device 116 and an output device 118. The input device 116 accepts user input, such as data related to carbohydrate consumption or other information regarding an individual, and the output device 118 provides information to the user, such as warnings, confirmation of user input data, current and historical blood glucose levels, operational modes or presumptions or recommendations, for example. In some embodiments, the controller 106 may be coupled to a database 120, such that the controller 106 is configured to selectively update the database 120 based on indications received from the sensor 102 and to selectively generate control signals to cause the dispenser 104 to dispense insulin, for example, to the patient 108.

The dispenser 104 may be an insulin pump, for example, that may be about the size of a small cell phone, for example, that is worn externally and can be discreetly clipped to the patient's belt, slipped into a pocket, or hidden under the patient's clothes. The dispenser 104 delivers both basal and bolus doses of insulin to match the body's needs. At the basal rate, the dispenser 104 delivers small amounts of insulin continuously for normal functions of the body (not including food). The bolus does is delivered by the dispenser 104 as additional insulin delivery “on demand” to match the food the patient anticipates eating or to correct a high blood sugar.

In general, the dispenser 104 includes a pump 122, a reservoir 124, an infusion set 126, and flexible tubing 128. The pump 122 may be an insulin pump defined by a housing that includes the reservoir 124. The reservoir 124 may be a plastic cartridge, for example, that holds the insulin and is locked into the insulin pump 122. In some embodiments, the cartridge includes a transfer guard that assists with pulling the insulin from a vial into the reservoir 124. The reservoir 124 can hold up to 300 units of insulin, for example, and is changed every two to three days. The infusion set 126 includes the flexible tubing 128 that goes from the reservoir 124 to an infusion site on the patient's body. A cannula is inserted with a small needle that is removed after it is in place and goes into sites (areas) on the patient's body similar to where one would give insulin injections. The infusion set 126 may be changed every two to three days.

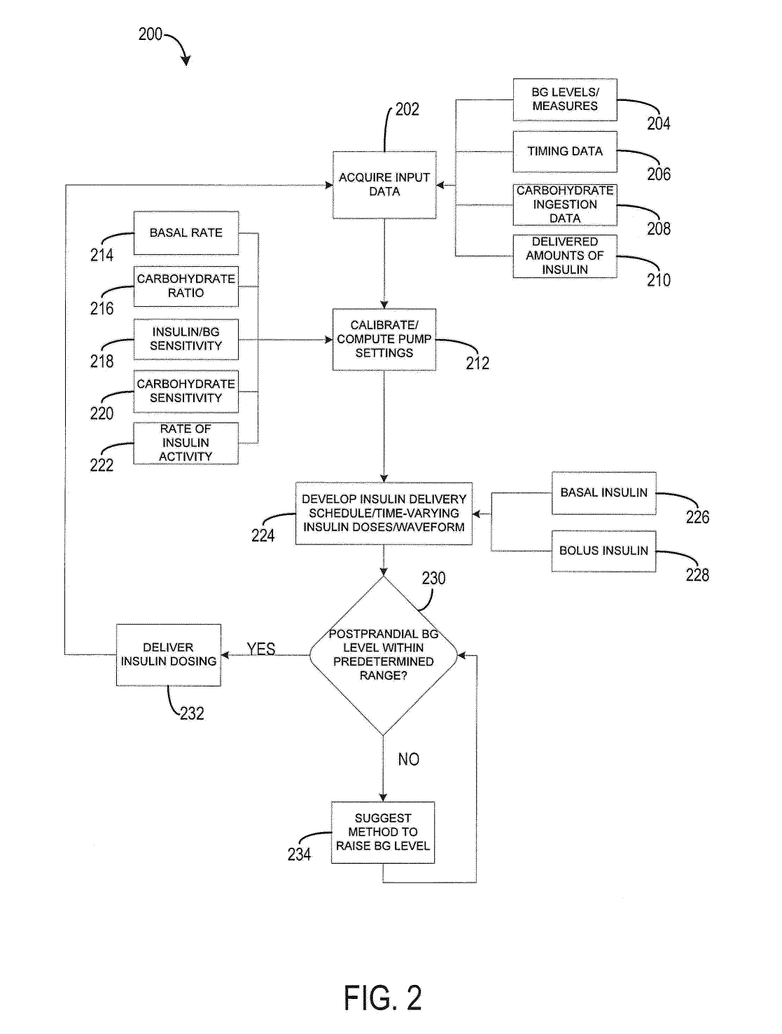

Turning now to FIG. 2, a flow chart setting forth exemplary steps 200 of a method for administering a pharmaceutical to an individual through a computed delivery schedule is shown. To begin the process, input data is acquired at process block 202. In some embodiments, the input data is acquired from the input device 116 and/or the sensor(s) 102 shown in FIG. 1. Input data acquired from the input device 116 may be acquired from input by the patient 108 and may include, for example, timing data, as shown at process block 206, carbohydrate ingestion data, as shown at process block 208, and delivered amounts of insulin, as shown at process block 210. Additionally, or alternatively, blood glucose levels, as shown at process block 204, may be acquired from the sensor(s) 102 of the dispenser control system 100. Ultimately, the input data acquired at process block 202 may be used to calibrate the pump (e.g., the pump 122 of the dispenser control system 100) settings at process block 212 to provide a simple to use, automated, individualized, insulin pump calibration system. As such, the input data acquired at process block 202 may be used to evaluate the impact of an algorithm, as will be described in further detail below, on the patient's 108 average blood glucose level.

The input data acquired at process block 202 is acquired before the patient 108 ingests carbohydrates and again after the carbohydrates have been metabolized (e.g., a few hours later). In one non-limiting example, dense measurements of intravenous blood glucose levels may be obtained every 10 minutes immediately before and for four hours after the administration of 33 grams of carbohydrates. The rate at which carbohydrates are metabolized is a food specific parameter, and thus will be different for a glass of juice than a slice of pizza, for example. However, the controller 106 may be configured such that the input data received by the input device 116 can be discretized into some small number of values, for example, 1-5, where 1 indicates rapid-acting carbohydrates and 5 indicates slow acting carbohydrates with high fat and/or protein content.

In some embodiments, the patient 108 may be prompted to indicate whether a blood glucose measurement 204 that has been entered into the input device 116 should be used for calibration of the pump settings at process block 212. For example, the output device 118 of the dispenser control system 100 may provide the user communication with a stand-alone software package built with either a web-interface, or run locally, to allow users of insulin pens and syringes to enter a series of measurements and have their basal and bolus rates computed.

Once the input data is acquired at process block 202, the controller 106 may be configured to execute the stored program to calibrate the pump settings at process block 212. The measurements of blood glucose levels 204 made by the pump user, in addition to delivered amounts of insulin 210 acquired at process block 202, may be utilized to accurately and automatically determine the correct settings for the insulin pump 122 at process block 212. The pump settings computed at process block 212 may include, but are not limited to, basal rate, as shown at process block 214, carbohydrate ratio, as shown at process block 216, insulin sensitivity, as show at process block 218, carbohydrate sensitivity, as shown at process block 220, and the rate of insulin activity, as shown at process block 222.

In one non-liming example, the same technique may be applied to Type I and Type II patients who use injections instead of pumps to accurately compute basal rates, carbohydrate ratios and insulin sensitivities, for example. As such, the pump auto-calibration process performed at process block 212 may automatically calibrate (and recalibrate) insulin dosing for Type I diabetes patients, as well as patients with Type II that require injected insulin.

In order to calibrate the pump setting at process block 212, the controller 106 of the dispenser control system 100 may be configured to execute a stored program including a non-limiting example algorithm, as shown in equation (1) below, to compute the relevant pump parameters (i.e., basal rate 214, carbohydrate ratio 216, insulin sensitivity 218, carbohydrate sensitivity 220, and the rate of insulin activity 222) from the input data acquired at process block 202. The basal rate 214 balances the rate at which the liver drips glucose into the bloodstream. The carbohydrate ratio 216 represents how many carbohydrates are accounted for by 1 unit of insulin. Insulin sensitivity or blood glucose sensitivity 218 is the amount the blood glucose levels drop in response to 1 unit of insulin, and carbohydrate sensitivity 220 may be derived from the carbohydrate ratio 216.

Ĝᵢ¹=Gᵢ⁰−S_{I}(Iᵢ+eΔtᵢ)+S_{C}Cᵢ,[S_{I},S_{C},e]=arg min Σᵢ₌₁^{M}(Ĝᵢ¹−Gᵢ¹)² (1)

The automated insulin pump calibration algorithm described in equation (1) above takes M pairs of blood glucose measures {{G₁⁰, G₁¹}, {G₂⁰, G₂¹} . . . {G_{M}⁰,G_{M}¹}} together with information on carbohydrate ingestion C_{i . . . M} and doses of insulin delivered I_{i . . . M}, and computes optimal basal adjustment e, insulin sensitivity S_{I}, carbohydrate sensitivity S_{c}, as well as estimates the carbohydrate ratio, which is defined as S_{c}/S_{I}.

The parameter vector is low enough dimensionally that a global search over discretized values of [S_{I}, S_{c}, e] is used to estimate the desired parameters. In an alternative embodiment, b-splines may be used to allow [S_{I}, S_{c}, e] to vary over the course of a day, replacing the squared error function with a robust m-estimator, such as a Tukey biweight, to be robust to the presence of unmetabolized, slow-acting carbohydrates.

Calibration of the pump settings at process block 212 may be simplified by looking at the set of measurements of the blood glucose levels 204 acquired at process block 202 after the set of carbohydrates have been metabolized by the patient 108. Thus, as previously described, the initial blood glucose level 204 is measured, the patient 108 is given a known amount of carbohydrates, and the blood glucose level 204 is measured multiple times a couple of hours later. The shape of the curve fit to those data points allows the proper basal rate 214 to be computed at process block 212, as well as the appropriate carbohydrate ratio 216 using standard parameter estimation techniques, such as min squared error.

Once the pump settings are calibrated at process block 212, the controller 106 is configured to execute the stored program to compute a delivery schedule based on the pump settings at process block 224. The delivery schedule may be a time-varying insulin delivery schedule that is characterized by a waveform, other than a conventional square-wave, to deliver both basal insulin, as shown at process block 226 and bolus insulin, as shown at process block 228.

Conventionally, each basal rate comprises a different insulin delivery with distinct start times and stop times characterized by a square waveform. Together, the different insulin rates cover a 24-hour period and are repeated each day. Example basal rate increments include 0.025 units for rates between 0.025-0.975 u/h, 0.05 units for rates between 1-9.95 u/h, and 0.1 units for rates of 10 u/h or more. However, these dosage forms are not optimal for controlling postprandial blood glucose, which is the blood glucose level taken approximately two hours after a meal and used to see if someone with diabetes is taking the right amount of insulin. These waveforms for delivering insulin doses also ignore an important advantage that insulin pumps possess over injections. That is, insulin pumps have the freedom to modulate the amplitude and scheduling of insulin delivery.

Thus, the time-varying insulin delivery schedule developed at process block 224 overcomes these drawbacks by delivering insulin doses and/or glucagon doses characterized by waveforms specifically adapted to the patient's needs. As such, the waveform of the time-varying insulin delivery schedule maybe characterized by varying amplitudes, frequencies, periods, and shape (e.g., sinusoidal, complex, ramped, etc.) to help improve (i.e., minimize) postprandial blood glucose levels from a preset target in the normal blood glucose level range. The optimization may use both the bolus insulin 228 (taken at meal time to keep blood glucose levels consistent after a meal) computed from the carbohydrate ingestion data 208 acquired at process block 202 that the user is intending to ingest, as well as the basal insulin 228 (keep blood glucose levels consistent during periods of fasting) that would ordinarily have been given over the time window.

In order to develop the time-varying insulin delivery schedule, a second non-limiting example algorithm, as shown in equation (2) below, may be integrated into the controller 106 of the dispenser control system 100. From the input data (e.g., carbohydrate ingestion data 208 and timing data 206) acquired from the patient 108 at process block 202, the algorithm can compute and deliver the optimal insulin (and potentially glucagon) waveform by explicitly solving for a time-varying set of insulin doses I(t) that minimize the excursions of the blood glucose levels Gₚ(t) from the normal range:

\hat{I}(t)=arg min ∫ₜ₌₀^{N}ƒ(Gₚ(I(t))=G_{T})dt,I(t)>=0∀t,∫ₜ₌₀^{N}I(t)dt=D,ƒ(x)=x² (2)

The model for Gₚ(t) is a differential model, similar to the model shown in equations (3) and (4) below:

Ġₚ=S_{c}k₁Cₛ+k₂−S_{I}k₄Iₚ, Ċₛ=−k₁Cₛ+c(t) (3)

İₚ=k₃Iₚ−k₄Iₚ+k₅I_{ƒ}, İ_{ƒ}=−k₅I_{ƒ}+k₃Iₚ+I(t) (4)

Where k₁ is the rate at which carbohydrates are metabolized, k₂ is the rate at which the liver supplies glucose into the plasma (basal=k₂/S_{I}), k₃ is the rate at which insulin moves from plasma to interstitial fluid, k₄ is the rate at which insulin transports glucose out of the plasma, k₅ is the rate at which insulin moves from interstitial fluid to plasma, c(t) is the time course of carbohydrates ingested, I(t) is the time course of insulin introduced into interstitial fluids, G_{T} is the target blood glucose level, Gₚ is the blood glucose level, Cₛ is the carbohydrate in the stomach, and D is the total insulin dose (basal*N+carbs/carb ratio+(Gₚ(t₀)−Gₜ)/S_{I}).

Equations (2), (3), and (4) may be simplified in that a number of the parameters can be inherited from equation (1) and do not need to be re-estimated. In one embodiment, equation (2) can be minimized using a variation of the Earth Mover's Problem (EMP), which has the advantages that total insulin is conserved, and the insulin dose is naturally constrained to be non-negative (a constraint that can be removed to simulate the use of a bihormonal pump). In other embodiments, forms of the function ƒ may be explored, as the EMP technique is quite general and does not require a quadratic form or even continuity. For example, ƒ could provide no penalty for blood glucose levels within a specified range (e.g., [90,140]), large penalties for blood glucose levels approaching the lower limit, more moderate penalties for moderate highs and again larger penalties for extremely high blood glucose levels.

Also, when developing the time-varying insulin delivery schedule at process block 224, the controller may be configured to determine what the effect of extending a pre-meal time window on average blood glucose may be. Additionally, or alternatively, the controller may be configured to determine what the effects of mis-estimating total carbohydrates or timing of carbohydrate intake may be. Such considerations may be important to ensure that small delays in mealtime do not result in dangerously low blood glucose levels for the patient 108.

To minimize postprandial blood glucose levels, the algorithms may be used to predict the blood glucose response to carbohydrate ingestion and insulin delivery and compute what schedule of insulin would keep the blood glucose in the target range. To accomplish this, an L1 or L2 norm may be used to penalize departures from a target blood glucose (e.g., 120). These can be solved directly for optimal solutions using linear or quadratic programming. Additionally, or alternatively, a Bayesian decision theory may be used to impose an additional “risk” function on top of the blood glucose to account for the fact that the risk of low and high blood glucose levels is asymmetric (e.g., being 80 points above target for a brief period of time is acceptable, while being 80 points below target is life-threatening). This would yield a different insulin schedule that can be computed using the solution from the L1/L2 approach described above as a starting point, then using techniques such as Markov-chain Monte-Carlo sampling.

Once the time-varying insulin delivery schedule is developed at process block 224, the controller may be configured to determine if the patient's postprandial blood glucose level is within a predetermined range at decision block 230. If the patient's postprandial blood glucose level is not within the predetermined range at decision block 230, the system may suggest, for example, how many carbohydrates are required to raise a low blood glucose level to the normal range within a certain time period. If, however, the patient's postprandial blood glucose level is within the predetermined range at decision block 230, the controller may be configured to deliver the time-varying insulin dose schedule to the patient 108 at process block 232. The process continues at process block 202 by acquiring input data at process block 202 and continually adjusting the pump settings at process block 212 and developing the delivery schedule at process block 224 to meet the patient's 108 needs.

### Example

The following Example is provided in order to demonstrate and further illustrate certain embodiments and aspects of the present invention and is not to be construed as limiting the scope of the invention.

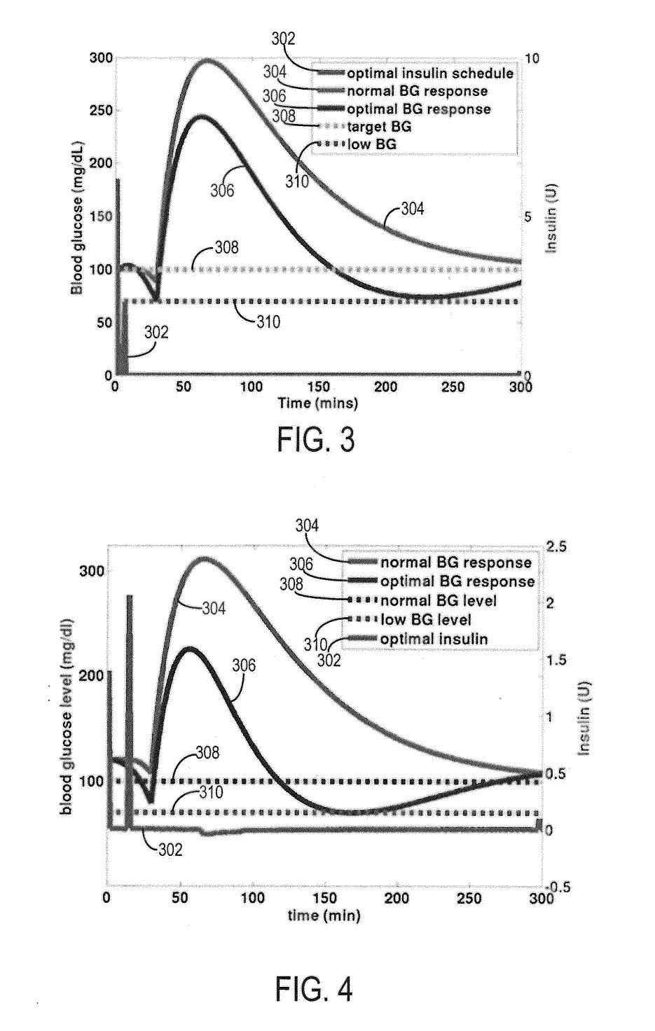

Turning now to FIGS. 3 and 4, graphs illustrating a blood glucose response to the infusion of insulin and glucagon, respectively, followed by ingestion of carbohydrates according to the computed delivery schedule are shown.

A preliminary simulation of the administration of a calibrated dose of insulin 302 at t=15 minutes before the ingestion of 50 grams of carbohydrates at t=30 minutes was run using the model and algorithms described above. The model was run twice, once with a non-negativity constraint (see FIG. 3) for insulin dose, and once removing this constraint to simulate the effects of a bi-hormonal pump and the infusion of soluble glucagon (see FIG. 4).

The average blood glucose level over the 5 postprandial hours for the blood glucose response 304 to the 15 minute pre-bolus is 180 mg/dL. It is worth noting that moving the insulin bolus 302 significantly further back in time runs the risk of hypoglycemia, and thus is not a viable option. In contrast, the insulin schedule 302 takes some of the basal insulin that would ordinarily have been delivered over the 5 hour window and moves it to an earlier time, significantly reducing average postprandial blood glucose levels to 124 mg/dL while not resulting in any hypoglycemic events. Having the flexibility of infusing glucagon (see FIG. 4) allows insulin/glucagon schedules 302 that reduce the average postprandial blood glucose levels even further to 112 mg/dL. If no prebolus time is allowed, the algorithm produces approximately the same average blood glucose reduction (about 50 mg/dL), although from a higher baseline.

More specifically, simulation of blood glucose response 302 to the infusion of insulin 302 followed 15 minutes later by the ingestion of 50 grams of carbohydrates is shown in FIG. 3. The optimal insulin delivery schedule 302 results in the optimal blood glucose response 306 with significantly lower average postprandial blood glucose (124 mg/dL). As shown in FIG. 4, further reductions (100 mg/dL) in postprandial blood glucose can be obtained using glucagon, where the target blood glucose level 308 is 100 mg/dL, and the low blood glucose level 310 is 70 mg/dL that the algorithm is constrained to stay above.

Thus, the invention provides systems and methods for automatically calibrating insulin pumps, developing time-varying insulin dosing schedules, and automatic minimization of postprandial blood glucose levels.

What is claimed is:

1. A method for administering a pharmaceutical to an individual through a dispenser control system, the method comprising:
acquiring input data from an input device coupled to a controller, the controller in communication with the input device and configured to execute a stored program to:
calibrate pump settings of the dispenser control system based on the acquired input data;
compute a delivery schedule based on the pump settings for the individual; and
activate the dispenser control system to deliver at least one dose of the pharmaceutical according to the delivery schedule, wherein the delivery schedule is characterized by a waveform other than a square-wave.

2. The method of claim 1 wherein the delivery schedule is determined based on a basal rate.

3. The method of claim 1 wherein the delivery schedule is determined based on a carbohydrate ratio.

4. The method of claim 1 wherein the delivery schedule is determined based on an insulin sensitivity.

5. The method of claim 1 wherein the delivery schedule is determined based on a carbohydrate sensitivity.

6. The method of claim 1 wherein the delivery schedule is determined based on carbohydrate ingestion data.

7. The method of claim 1 wherein the delivery schedule is determined based on an amount of insulin delivered to the individual.

8. The method of claim 1 wherein the pharmaceutical to be delivered to the individual includes insulin.

9. The method of claim 1 wherein the controller executes the program stored in the controller to activate the dispenser control system to deliver the at least one dose of the pharmaceutical to the individual when the at least one of the pump settings and the input data is outside a predetermined threshold, the predetermined threshold includes at least one of a target blood glucose level of the individual and a postprandial blood glucose level of the individual.

10. The method of claim 1 wherein the input data includes at least one of blood glucose levels of the individual, timing data, carbohydrate ingestion data, and delivered amounts of insulin.

11. The method of claim 1 wherein the pump settings are based on at least one of a basal rate, a carbohydrate ratio, an insulin sensitivity, a carbohydrate sensitivity, and a rate of insulin activity.

12. The method of claim 11 wherein the controller executes the program stored in the controller to estimate the basal rate and the carbohydrate ratio using a minimum squared error estimation technique.

13. The method of claim 1 wherein the at least one dose of the pharmaceutical includes basal insulin and bolus insulin.

14. The method of claim 1 wherein the controller executes the program stored in the controller to recommend a quantity of carbohydrates for the individual ingest to maintain the at least one of the pump settings and the input data according to the delivery schedule.

15. A dispenser control system for administering a pharmaceutical to an individual, the dispenser control system comprising:
an input device coupled to a controller for receiving input data from the individual; and
a dispenser in communication with the controller, the dispenser including a pump for delivering the pharmaceutical from a reservoir to the individual through a tubing coupled to the individual;
wherein the controller is configured to execute a program stored in the controller to:
calibrate pump settings of the dispenser based on the acquired input data;
compute a delivery schedule based on the pump settings for the individual; and
activate the dispenser control system to deliver at least one dose of the pharmaceutical according to the delivery schedule, wherein the delivery schedule is characterized by a waveform other than a square-wave.

16. The dispenser control system of claim 15 wherein the pharmaceutical to be delivered to the individual includes insulin.

17. The dispenser control system of claim 15 wherein the controller executes the program stored in the controller to activate the dispenser control system to deliver the at least one dose of the pharmaceutical to the individual when the at least one of the pump settings and the input data is outside a predetermined threshold, the predetermined threshold includes at least one of a target blood glucose level of the individual and a postprandial blood glucose level of the individual.

18. The dispenser control system of claim 15 wherein the input data includes at least one of blood glucose levels of the individual, timing data, carbohydrate ingestion data, and delivered amounts of insulin.

19. The dispenser control system of claim 15 wherein the pump settings are based on at least one of a basal rate, a carbohydrate ratio, an insulin sensitivity, a carbohydrate sensitivity, and a rate of insulin activity.

20. A method for administering a pharmaceutical to an individual through a dispenser control system, the method comprising:
acquiring input data from at least one of a sensor coupled to the individual and an input device coupled to a controller, the controller in communication with the sensor and configured to execute a stored program to:
calibrate pump settings of the dispenser control system based on the acquired input data;
compute at least one dose of the pharmaceutical to be delivered to the individual through the dispenser control system; and
activate the dispenser control system to deliver the at least one dose of the pharmaceutical to the individual when at least one of the pump settings and the input data is outside a predetermined threshold.
